(19) United States
(12) Patent Application Publication
(10) Pub. No.: US 2016/0045647 A1
(43) Pub. Date: Feb. 18, 2016

(54) Liquid-accommodating container for a device for providing a vacuum for medical applications, method for producing the container, and device

(71) Applicant: PAUL HARTMANN AG, Heidenheim (DE)

(72) Inventors: Pierre Croizat, Herbrechtingen (DE); Axel Eckstein, Heidenheim (DE); Juana Kirsten, Langenau (DE); Astrid Fonfara-Doerr, Ulm (DE); Andreas Mahler, Blaubeuren-Asch (DE)

(21) Appl. No.: 14/784,291

(22) PCT Filed: Apr. 29, 2014
(86) PCT No.: PCT/EP2014/058691

§ 371 Date: Oct. 14, 2015

(30) Foreign Application Priority Data
May 3, 2013 (DE) 10 2013 208 108.1

(51) Int. Cl. A61M 1/00

(52) U.S. Cl. CPC A61M 1/0001; A61M 1/0088

(57) ABSTRACT

A liquid-accommodating container (8) for a device (2) providing vacuum for medical applications, in particular for the vacuum treatment of wounds in a human or animal body. The container (8) has two half-shell parts (20, 22), which are permanently joined to each other and bound the interior of the container in the joined state. A filter (100) lies against an inner region (106) of the half-shell part (22) facing away from a first housing part (4) on one side and against a sealing element (54) on the other side under axial stress in an axial direction (102). The sealing element (54) is attached to the half-shell part (20) facing the first housing part (4), wherein the axial stress is formed by the joining of the half-shell parts (20, 22) and associated deformation of the sealing element (54) by the filter (100).

This application is the national stage of PCT/EP2014/058691, filed Apr. 29, 2014 and claims Paris convention priority from DE 10 2013 208 108.1, filed May 3, 2013.

### BACKGROUND OF THE INVENTION

The invention relates to a liquid-accommodating container for a device, which can be worn on the body of a user and additionally operated in a stationary manner, for providing a vacuum for medical applications, in particular for vacuum treatment of wounds in the human or animal body, wherein the device comprises a first housing part having a vacuum-generating apparatus and the container, which can be discarded after use, for collecting bodily fluids in the interior of the container, in particular wound secretions suctioned from a wound, wherein the container comprises a second housing part of the device, which can be manually fastened to and manually detached from the first housing part of the device and, when in the fastened state, a vacuum can be applied to the interior of the container from the vacuum-generating apparatus, and wherein a connection is provided on the container for a suction line leading to the body, such that a vacuum communication can be produced between the vacuum-generating apparatus, the container and the suction line leading to the body, wherein the container comprises two half-shell parts, which are permanently joined to each other and bound the interior of the container in the joined state, wherein a region for accommodating a filter is also formed in the interior of the container.

Vacuum treatment means that a region of the body or wound exposed to the ambient atmosphere is sealed in a pressure-tight or vacuum-tight manner from the environment, i.e. the atmosphere in which we live and breathe, by means to be described in more detail, wherein, within the sealed wound region, a pressure that is lower than the atmospheric pressure, therefore a vacuum relative to the atmosphere, can be applied and permanently maintained. When we speak of a vacuum in this context, we mean a pressure range that is typically between 0 and 250 mmHg (millimeters of mercury) below the surrounding atmospheric pressure. This has been shown to be beneficial to wound healing. For the vacuum-tight sealing, a vacuum dressing is typically used, which can comprise, for example, a pressure-tight or vacuum-tight layer of film, which is typically glued onto an unaffected region of the body surrounding the wound, so that a tight seal can be achieved.

The object of this invention is to accommodate and mount a filter in an economic manner and also to ensure safety of operation.

### SUMMARY OF THE INVENTION

This object is inventively achieved in a liquid-accommodating container of the type stated above in that the filter lies against an inner region of the half-shell part facing away from the first housing part on one side and against a sealing element on the other side under axial stress in an axial direction extending approximately perpendicularly to a dividing plane of the half-shell parts, which sealing element is attached to the half-shell part facing the first housing part, wherein the axial stress is formed by the joining of the half-shell parts and associated deformation of the sealing element by the filter.

Because the filter is accommodated under axial stress between the two half-shell parts, secure positional fixture of the filter within the container is achieved. Due to the axial pressure, secure sealing between the filter and the sealing element is also achieved. Dimensional tolerances in the filter can be compensated or secure sealing achieved despite dimensional tolerances. The filter can be easily mounted because the filter can be disposed between the half-shell parts before they are joined. It is also conceivable for the filter to be inserted through a correspondingly large opening in a wall of the half-shell part of the container facing the first housing part and then positionally fixed by disposing the seal in this opening.

In an advantageous further embodiment of the invention, the filter preferably lies against multiple ribs that are constituted in the interior of the half-shell part facing away from the first housing part. Compared to seating over a large surface area, this has the advantage that a larger active filter surface can be exposed to the remaining interior space, that is, the liquid-accommodating space of the container.

The filter itself can be, for example, block or disk-shaped. It proves advantageous if the filter is constituted cylindrically, pot-shaped, conically, or as a tapered cylinder because this provides a larger penetration area of the filter. Irrespective of the specific shape of the filter, it is advantageously made of a rigid and dimensionally stable material. Suitable filter materials include, for example, porous plastic solids (for example, polyethylene or polypropylene), ceramics or metal.

Admixture of an expandable substance (for example, carboxymethyl cellulose) can also be provided, which closes the pores in the filter material after contact with liquid, thus preventing penetration of further liquid through the filter. Admixture of an odor-absorbing substance (for example, active carbon), which retains gaseous substances, which can cause a olfactory nuisance, is also advantageous. The odor-absorbing substance can also be a coating.

The filter is advantageously accommodated in a chamber that is bounded in the interior of the container by a closed wall. This wall can advantageously have one or more openings, by which it communicates in a fluid-dynamic connection with the remaining interior of the container or with a splash-water-protected antechamber.

It also proves advantageous if the sealing element is an injection-molded part made of elastically compliant material, which is retained, preferably by a positive connection, in an opening in a wall of the first half-shell part of the container. The sealing element can, for example, be glued on or clipped in or fixed in its operating position by means of further mounting elements.

In a further advantageous embodiment of the invention, the sealing element is an injection-molded part made of elastically compliant material, which is disposed in an opening in a wall of the first half-shell part of the container and overmolded onto the wall. By overmolding the sealing element it is also possible to achieve a positively connected back grip.

In a preferred embodiment of the invention, the sealing element has an axial opening.

It further proves advantageous if the sealing element is constituted concentrically with respect to the axial direction.

It further proves advantageous if the sealing element comprises a step with respect to the axial direction against which the filter rests and which is preferably constituted radially in the interior.

It further proves advantageous if the sealing element has a tubular projection that projects into the interior of the container, in which the filter engages. This embodiment has the advantage that the filter can be inserted into the tubular projection during mounting so that it can be positioned in its correct mounting position, in particular by a frictional connection.

It further proves advantageous if the sealing element has a tubular projection that projects from the container toward the first housing part, by means of which a fluid-dynamic connection can be created to the vacuum-generating apparatus. The sealing element thereby has a dual function: It seals the filter in the container toward the outside and it also forms an interface for fluid-dynamic communication with the vacuum-generating apparatus in the first housing part.

In a further embodiment of the invention, it is proposed that a further sealing element be provided, which is disposed between an, in particular closed off chamber in the interior of the container and a medium or measuring connection in the first housing part and which projects from the container toward the first housing part with a tubular projection. This further sealing element can basically be manufactured and be disposed in the same way as the sealing element assigned to the filter. It can, in particular, be overmolded directly onto a wall of the container.

In this case, it can prove advantageous if the sealing elements are formed in the same injection-molding process and are connected at the container via a filling gate filled with elastomeric material.

The invention also concerns a device as well as a method for manufacturing a container, each in accordance with the respective dependent or independent claim.

Further characteristics, details, and advantages of the invention result from the attached claims and from the drawings and description below of a preferred embodiment of the invention.

The drawing shows:

### BRIEF DESCRIPTION OF THE DRAWING

FIGS. 1a, b a perspective view of a device for providing a vacuum for medical applications with a container, which can be discarded after use, for collecting bodily fluids;

FIG. 2 a perspective view of a half-shell of the container according to FIG. 1 viewed from the inside;

FIG. 3 a perspective view of the half-shell according to FIG. 2 viewed from the outside;

FIG. 4a the half-shell according to FIG. 3 with overmolded sealing elements;

FIGS. 4b, c two views of the overmolded sealing elements;

FIG. 5 the other half-shell of the container according to FIG. 1 viewed from the inside;

FIG. 6 the half-shell according to FIG. 5 viewed from the outside;

FIGS. 7a, b sectional views of the container.

### DESCRIPTION OF THE PREFERRED EMBODIMENT

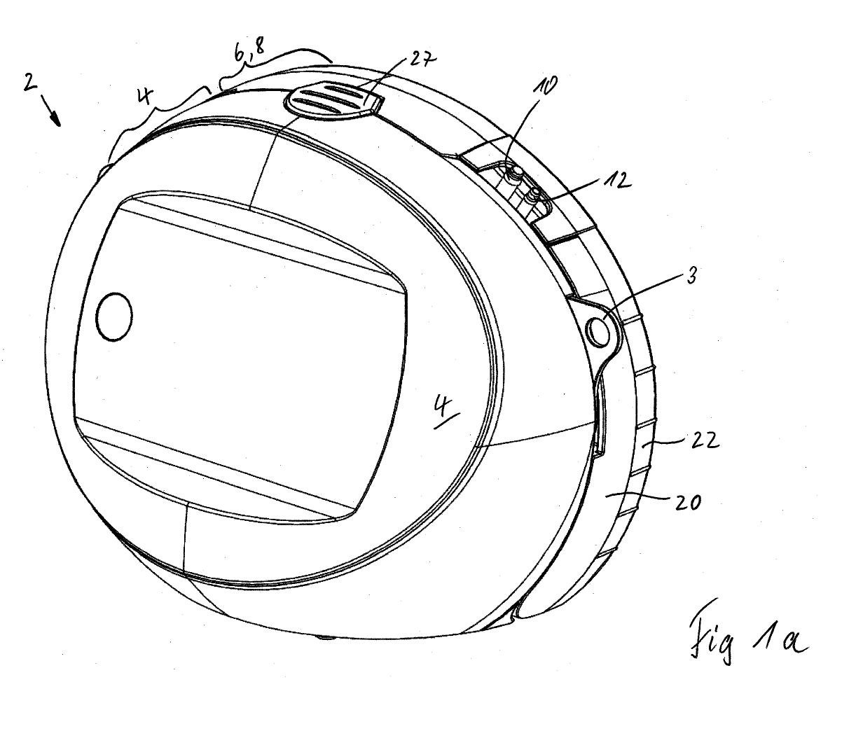

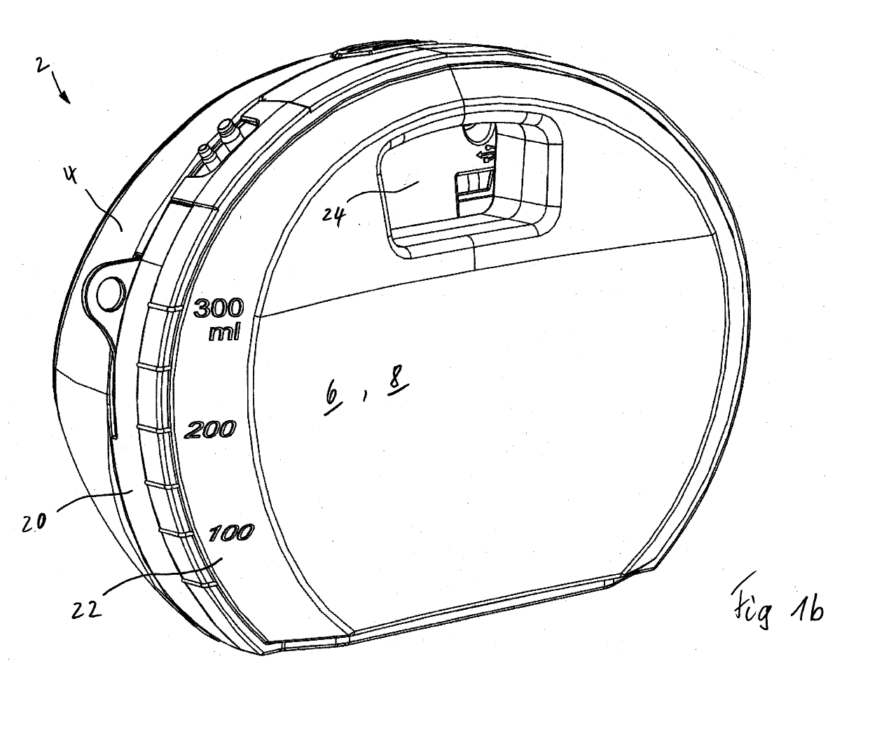

FIG. 1a, b shows a device collectively designated with reference number 2 for providing a vacuum for medical applications. The device 2 can be worn on the body of a user (mobile operation), but can also be operated in a stationary manner (stationary operation). In mobile operation, the device could be carried by means of a strap or belt or similar and worn on the body, wherein for this purpose a fastening loop 3 is shown purely as an example. In stationary operation, the device can be placed on a preferably level surface. The device 2 comprises a first housing part 4, in which a vacuum-generating apparatus as well as electric or electronic control components for the device 2 as a whole are accommodated, including batteries or preferably rechargeable batteries. Moreover, the device 2 comprises a second housing part 6, which is constituted by a container 8 for collecting bodily fluids in its interior, in particular for collecting wound secretions suctioned out of a wound. The container 8 is constituted as an article, which can be discarded after use. The container 8 comprises upper connecting glands 10, 12 for a suction line (not depicted) and a line for feeding media toward the body of the patient or for forming a measuring duct. The suction line, which is not depicted, leads, in the exemplary operation of the device for the vacuum therapy of wounds, to a wound dressing that seals the wound in a pressure-tight manner and communicates at that location, for example via a port, with a wound region in order to create, maintain, or vary a vacuum in the wound region, and to suction wound exudates into the container 8. To achieve this, the container 8 communicates with the vacuum-generating apparatus in the first housing part 4.

The connecting gland 12 also leads via a tube (not depicted) which constitutes a measuring or rinsing duct, toward the body of the user, in particular, to a wound. The connecting gland 12 communicates, in a way to be described in more detail, with the first housing part 4 through the interior of the container 8 where a supplied medium, in particular a rinsing medium, can be added to it, or where it is connected to a measuring connection.

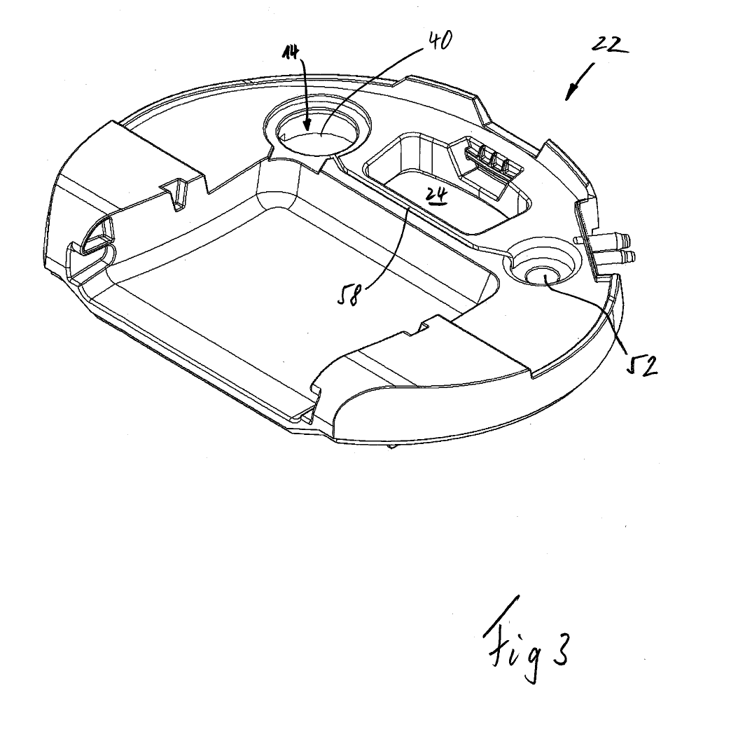

The connecting gland 10 for the suction line opens out into the interior of the container 8. The interior of the container 8 communicates via a vacuum interface 14 (FIG. 3, 4a), to be described in more detail, with the vacuum-generating apparatus within the first housing part 4 when the container 8 is fastened on the first housing part in the operating position (see FIG. 1). For this purpose, the container 8 can be positioned manually on the first housing part 4 and then brought manually into a mechanically fastened and mechanically detachable mounting position, in particular by latching. In this mounting position, vacuum communication between the interior of the container 8 and the vacuum-generating apparatus and fluid-dynamic communication between the connecting gland 12 and the assigned measuring or rinsing connection on the first housing part 4 is automatically achieved, in the manner basically described in DE 10 2009 038 130.9, the relevant disclosure of which is hereby incorporated by reference.

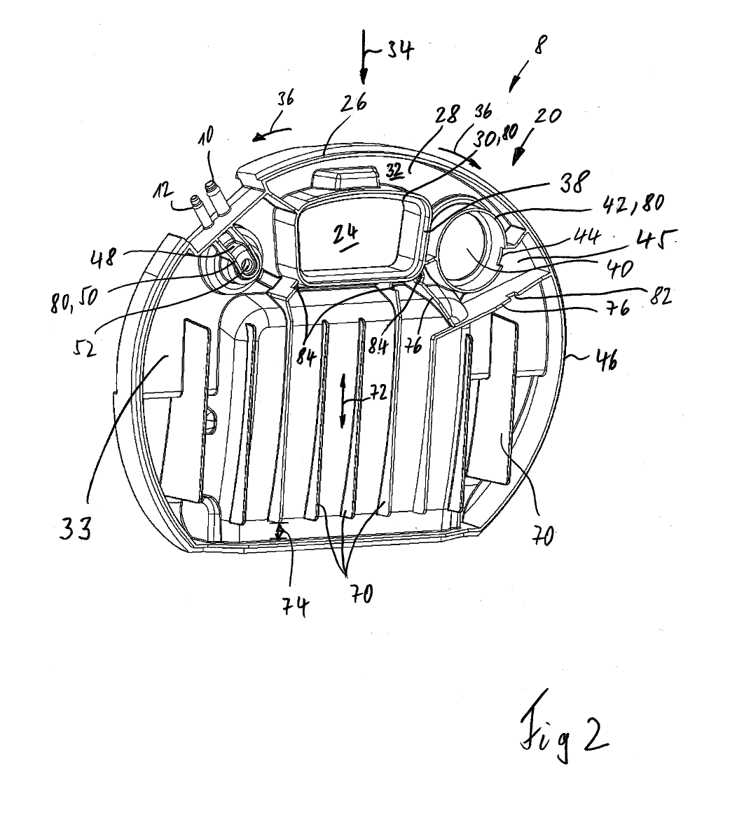

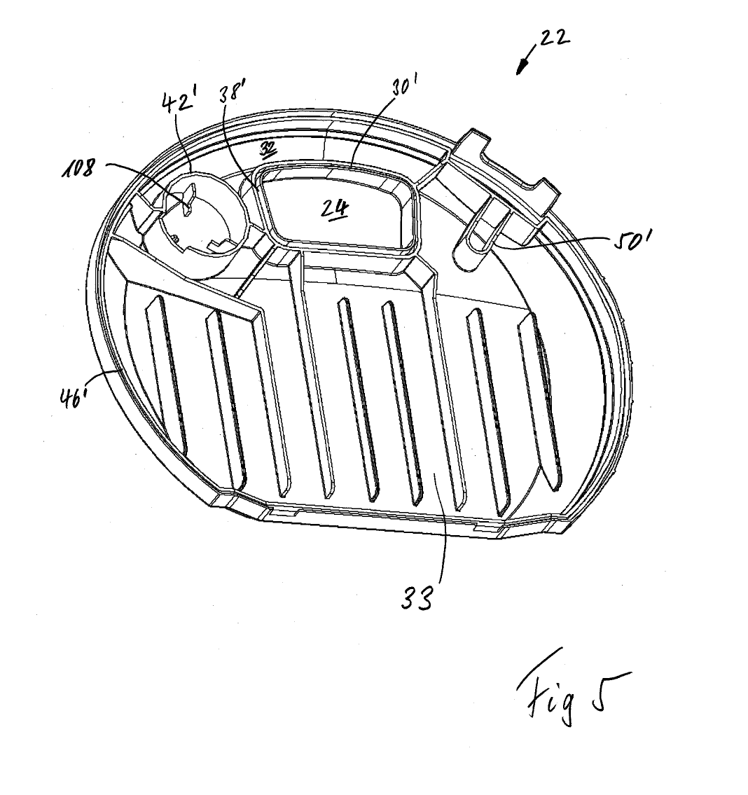

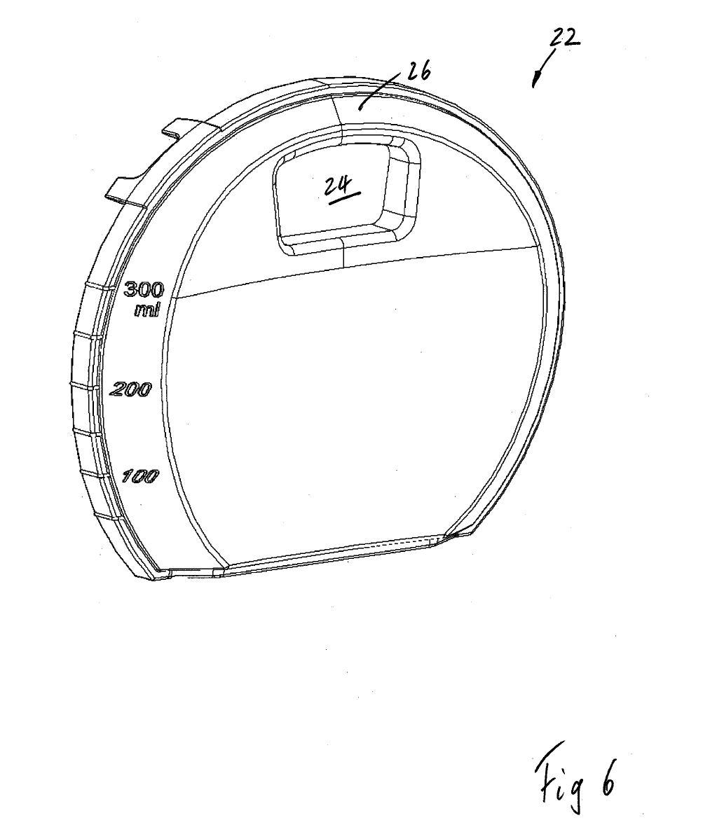

FIGS. 2 to 6 show the embodiment of the container 8 that forms the second housing part 6. The container 8 comprises a first half-shell part 20 that faces the first housing part 4 and a second half-shell part 22 that faces away from the first housing part 4 (FIGS. 5, 6) and can be placed on the body of the user in mobile operation, which, when assembled, bound the interior of the container 8. The container 8 also has a grip opening 24, which, in the illustrated example, is constituted as a grip-through opening that penetrates through the container 8 and which is formed and/or bounded by the two half-shell parts 20, 22. In the first container 8, the grip opening 24 is constituted approximately in an upper quarter and centrally, so that the center of gravity of the container and of the device as a whole is located approximately below the grip opening 24. The container 8 and also the entire device can be gripped by manual gripping in the grip opening 24 with the fingers of the user. At the same time, the user grips right round a relatively wide web 26, which forms a grip area, constituted above the grip opening 24 with his or her hand. When gripping round this web 26, the user can, preferably with the thumb, operate an unlocking organ 27 (FIG. 1a), so that the container is released from its operating position depicted in FIG. 1a at the first housing part 4 and can be lifted upward at an inclined angle. This is also described in DE 2009 038 130.9, the relevant disclosure of which is hereby incorporated by reference.

In the figures, it can be seen that the interior of the container 8 extends into a region 28 adjacent to a wall 30 that bounds the grip opening 24. In the example and illustrated preferred case, this region 28 is constituted above a wall 30 that upwardly bounds the grip opening 24. This region 28 is bounded or formed by a chamber 32, which is closed off, that is, partitioned off in such a manner that it does not communicate with other adjacent chambers or regions in the interior of the container 8. This means that a body fluid suctioned into a liquid collection chamber 33 in the interior of the container 8, in particular wound exudates suctioned from a wound, cannot penetrate into this chamber 32 even if the container 8 is tipped or briefly tilted. Thus, the chamber 32 forms a kind of visual protection in the region of the grip opening 24 and in the region of the grippable web 26, when viewed from outside the container 8. In the preferred case shown by way of example, this chamber 32 extends above the entire grip opening 24 as seen from above in the vertical direction 34. The chamber 32 also extends beyond the grip opening 24 in the direction of the arrows 36 toward two opposite sides. From FIG. 2 it can further be seen that the chamber 32 also extends adjacent to a wall 38, which laterally bounds the grip opening 24 and is bounded toward the outside by a wall 42, which forms a chamber 40 for accommodating a filter.

Unlike chamber 32, the chamber 40, which accommodates the filter, is not fluid-dynamically closed off. It communicates with the vacuum-generating apparatus in the first housing part 4 as well as with an antechamber 45 via a vacuum communicating opening 44 in the wall 42. In respect of its walls, the second half-shell part 22 is constituted perpendicular to the dividing plane in a complementary manner to the first half-shell part 20. FIG. 5 shows a wall 42′ constituted in a complementary manner to the wall 42. The wall 30, 38 that bounds the grip opening 24 and which extends through the entirety of one circumferential direction, as well as an outside wall 46 and the associated complementary walls 30′, 38′, 46′ are constituted in the separation plane in their joint region in such a way that at least one step of the joining ends is constituted there, which has a self-centering effect also makes it easier to seal and bond the half-shell parts 20, 22.

Starting from the vacuum-generating apparatus, a vacuum can be built up in the interior of the container 8 through the filter (not shown in FIGS. 2 to 6) and through the opening 44 in the wall 42. Because the connecting gland 10 opens out directly into the interior of the container 8, a vacuum can be applied to the wound via the suction line (not depicted) and bodily fluids can also be suctioned from there into the interior of the container 8.

As seen from the outside, the further connecting gland 12 opens into a further chamber 48 that is closed off from the remaining compartment of the container 8 and bounded by the walls 50 and 50′ respectively, closed in the circumferential direction, of the two half-shell parts 20, 22. A feed-through duct 52 leads from this chamber 48 in the depth direction, that is, perpendicular to the separation plane, toward the first housing part 4, that is, toward the outside of the container 8.

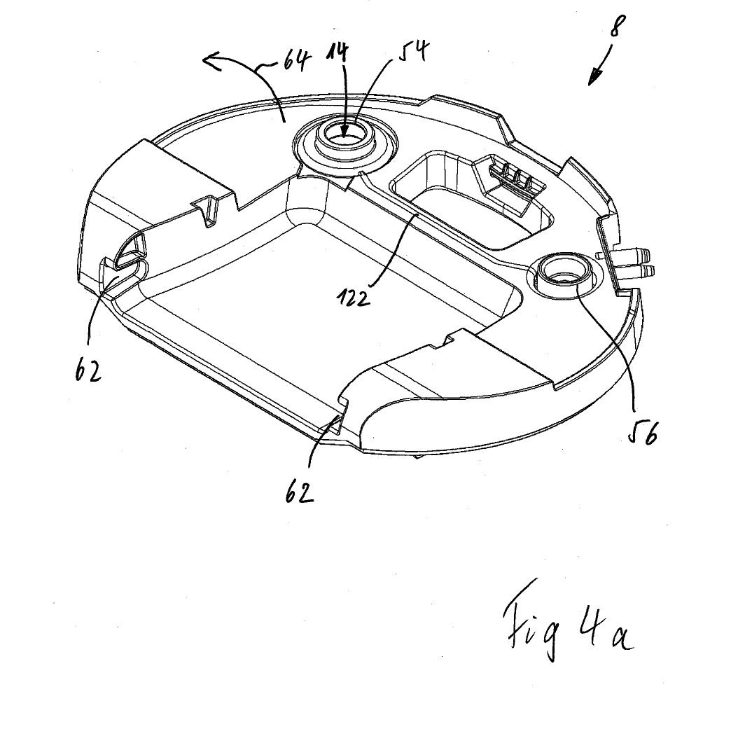

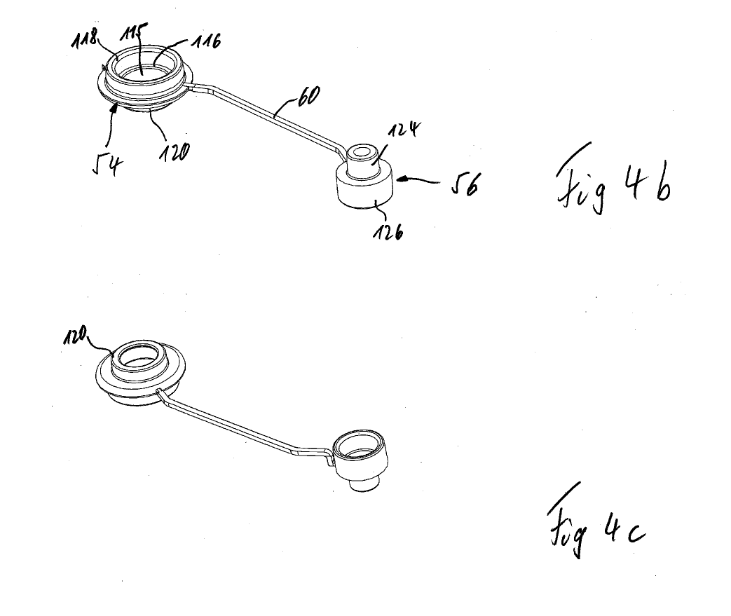

FIGS. 3 and 4 show the first half-shell part 20 from the outside, that is, looking onto the side facing the first housing part 4 in the operating position. FIGS. 3 and 4a show that a sealing element 54 and 56 can be preferably overmolded directly onto the first half-shell part 20 from the outside to seal the chamber 40 for the filter and to seal the feed-through duct 52. The narrow duct 58 is a supply channel made of injection-molded material, which forms a web 60 that connects both sealing elements 54, 56. As seen in FIG. 4a, the sealing elements 54, 56 have a concentric, cylindrical section that projects outward. When the container 8 is positioned on the first housing part 4, the container 8 is placed with two bearing areas 62 that open out at the bottom roughly in a U shape obliquely from above onto a mating bearing area on the first housing part 4 constituted in a complementary manner, and then pressed against the first housing part 4 in the direction of arrow 64, that is, roughly transversely with respect to the general disk shape of the container 8, wherein the two sides latch as mentioned above. Here, the cylindrical sections of the sealing elements 54, 56 are pressed against the preferably conical connection pieces that project from the first housing part 4, wherein a sealing fluid-dynamic connection is created.

The interior of the container 8 is partitioned by a multiplicity of ribs (see FIG. 2). Ribs 70 of a first type essentially extend within the liquid collection chamber 33 in the vertical direction 72, that is, in the direction of the arrow 34. They terminate at the bottom at a distance 74 of preferably at least 5 mm, so that the liquid can distribute itself evenly in the interior of the container when the container 8 is tilted and during regular suction of the liquid through the connecting gland 10.

Moreover, there are ribs 76 of a second type that extend obliquely. With these ribs, sloshing of the liquid collected in container 8 toward the filter chamber 40 is prevented. Such a rib 76 bounds the previously mentioned antechamber 45 to filter chamber 40.

For the purposes of vacuum communication, a relatively small opening 82 is constituted in the obliquely extending rib 76, which establishes vacuum communication between the antechamber 45 and the remaining interior of the container 8, but which provides great resistance to penetrating liquid, which could block the filter.

Furthermore, there are ribs 80 of a third type, which are joined to themselves in the circumferential direction. They form, for example, the wall 42 for the chamber 40 or the wall 50 for the chamber 48. The wall 30, which is joined to itself in the circumferential direction and which bounds the grip opening 24, can also be referred to as rib 80 of the third type.

The ribs 70, 76, which extend up as far as an inclined or horizontally extending wall also preferably have an opening 84 at the very top intended for equalizing pressure and allowing gases to pass, so that no air has to be led through the liquid.

The two half-shell parts 20, 22, are joined to each other permanently so that they form a seal preferably by gluing or thermowelding and thus constitute the second housing part 6 of the container 8 that constitutes device 2. The chamber 32, which is closed off from the liquid and the remaining interior of the container 8, protects the manually grippable web or the grip area 26 from contamination from bodily fluid collected in the interior of the container 8. It also provides visual protection, which, viewed from above, extends over the area of the grip opening 24 partially or preferably completely.

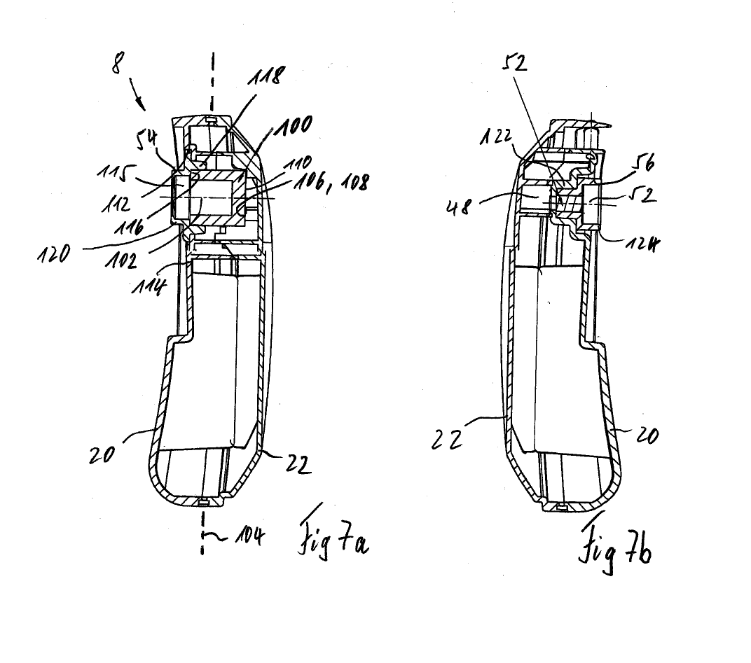

FIG. 7a shows the configuration of the filter 100 in the container 8. It can be seen that the filter 100 is disposed between the sealing element 54 and an internal area 106 of the half-shell part 22, in an axial direction 102, which also corresponds to the joint direction of the half-shell parts 20, 22 and extends perpendicularly to a dividing plane 104 between the half-shell parts 20, 22. This internal area 106 is preferably formed from a plurality of ribs 108, which are particularly clearly visible in FIG. 5. They also advantageously extend in the axial direction 102 and preferably additionally radially with respect to the axial direction 102. The filter 100 can, for example, be pot-shaped, wherein the outside of the pot base 110 rests axially against the ribs 108. The sealing element 54 is made of an elastically compliant material. It is disposed in an opening 112 in a wall 114 of the first half-shell part 20 of the container 8, and preferably by being overmolded directly at that position. The sealing element 54 is preferably constituted concentrically and comprises an opening 115 and an inside step 116 against which the filter 100 rests in the axial direction 102. This causes the sealing element 54 to deform slightly, which results in a clamping force in the axial direction 102 that holds the filter 100 in its intended mounting position. As can be seen in FIG. 7a and FIG. 4b, the sealing element 54 comprises a tubular projection 118 that projects into the interior of the container 8, into which the filter 100 engages axially. The internal diameter of the tubular projection 118 matches the outside circumference of the filter 100 in such a manner that the latter is fixed inside it by a clamping and frictional connection, which makes it easier to join together the half-shell parts 20, 24.

Moreover, the sealing element 54 comprises a tubular projection 120 that projects from the container 8 toward the first housing part 4, by means of which a fluid-dynamic connection can be created to the vacuum-generating apparatus in the first housing part 4. For example, the tubular projection 120 can be positioned against a, for example, conical projection so that it automatically seals and creates a fluid-dynamic connection when the container 8 is placed in its intended mounting position on the first housing part 4.

FIG. 7b shows a sectional view that corresponds to FIG. 7a but in the opposite viewing direction through the further sealing element 56, which is constituted as illustrated in FIGS. 4b, c. Like sealing element 54, it has tubular projections 122, 124. Its feed-through duct 52 opens into the chamber 48 mentioned above, which is sealed off from the remaining interior of the container 8 and communicates via the connecting gland 12 (FIG. 2) with a rinsing or measuring tube (not depicted).

Both sealing elements 54, 56, are preferably manufactured according to the same injection molding process and overmolded directly onto the first half-shell part 20. In order to have to provide no more than one gate in an injection mold to be used for this process, the two sealing elements 54, 56 are interconnected via a shared filling duct 126 and filled with elastomeric material accordingly (s. FIGS. 4).

1-16. (canceled)

17. A throw away liquid-accommodating container for a device, the device structured to be worn on a body of a user and also structured for operation in a stationary manner, the device generating a vacuum for medical applications or for vacuum treatment of wounds on a human or animal body, wherein the device has a first housing part accommodating a vacuum-generating apparatus, the throw away container being structured for collecting bodily fluids in an interior thereof or for collecting wound secretions suctioned out of a wound, the container comprising:
structure defining a second housing part that can be manually fastened to and manually detached from the first housing part, wherein, in a fastened state, the container is thereby structured to accommodate a vacuum applied to the interior of the container by the vacuum-generating apparatus;
a connection for a suction line leading to the body such that vacuum communication among the vacuum-generating apparatus, the container and said suction line is established;
two half-shell parts which are permanently joined to each other and bound the interior of the container in a joined state thereof;
structure defining a first region in the interior of the container;
a sealing element attached to a first half-shell part facing the first housing part; and
a filter disposed within said first region, wherein one side of said filter seats against an inner region of a second half-shell part facing away from the first housing part and an other side of said filter seats against said sealing element, said filter thereby being loaded by an axial stress in an axial direction extending approximately perpendicularly to a dividing plane of said half-shell parts, wherein said axial stress is formed by joining of said half-shell parts and associated deformation of said sealing element by said filter.

18. The container of claim 17, wherein said filter rests against ribs, which are formed in the interior of said second half-shell part.

19. The container of claim 17, wherein said filter is accommodated in a chamber that is bounded by a wall that joins onto itself.

20. The container of claim 17, wherein said filter is constituted cylindrically, pot-shaped or conically.

21. The container of claim 17, wherein said sealing element is an injection-molded part made of essentially elastically compliant material and is retained or is retained by a positive connection, in an opening in a first wall of said first half-shell part.

22. The container of claim 17, wherein said sealing element is an injection-molded part made of elastically compliant material and is disposed in an opening in a first wall of said first half-shell part and overmolded onto said first wall.

23. The container of claim 17, wherein said sealing element has an axial opening.

24. The container of claim 17, wherein said sealing element is constituted concentrically with respect to said axial direction.

25. The container of claim 17, wherein said sealing element comprises a step with respect to said axial direction against which said filter lies.

26. The container of claim 25, wherein said step is constituted radially in an interior region.

27. The container of claim 17, wherein said sealing element comprises a tubular projection that projects into the interior of the container and in which said filter engages.

28. The container of claim 17, wherein said sealing element comprises a tubular projection that projects from the container toward the first housing part and by means of which a fluid-dynamic connection is created to the vacuum-generating apparatus.

29. The container of claim 17, further comprising a further sealing element which is disposed between a chamber in the interior of the container and a medium or measuring connection in the first housing part, said further sealing element having a tubular projection which projects from the container toward the first housing part.

30. The container of claim 29, wherein said further sealing element is an injection-molded part made of elastically compliant material.

31. The container of claim 30, wherein said further sealing element is disposed in an opening in a wall of said first half-shell part and overmolded onto that wall.

32. The container of claim 29, wherein said sealing element and said further sealing element are formed in a same injection-molding process and are connected at the container via a filling gate filled with elastomeric material.

33. A method for manufacturing the container of claim 17, the method comprising the steps of;
a) providing the two half-shell parts;
b) overmolding the sealing element onto the first half-shell part;
c) disposing the filter on the sealing element or inserting the filter into the sealing element;
d) joining the two half-shell parts with interposition of the filter, thereby pressing the filter in an axial direction against the sealing element and creating a stress in the axial direction by joining the half-shell parts, with associated deformation of the sealing element by the filter; and
e) attaching the two half-shell parts to each other in a non-detachable manner.

34. A device structured to be worn on a body of a user and also structured for operation in a stationary manner, the device generating a vacuum for medical applications or for vacuum treatment of wounds on a human or animal body, the device comprising:
a first housing part accommodating a vacuum-generating apparatus;
two half-shell parts which are permanently joined to each other and bound an interior of a throw away liquid-accommodating container in a joined state thereof, said throw away container being structured for collecting bodily fluids in an interior thereof or for collecting wound secretions suctioned out of a wound;
structure defining a second housing part that can be manually fastened to and manually detached from the first housing part, wherein, in a fastened state, said container is thereby structured to accommodate a vacuum applied to the interior of the container by the vacuum-generating apparatus;
a connection for a suction line leading to the body such that vacuum communication among the vacuum-generating apparatus, the container and said suction line is established;
structure defining a first region in the interior of the container;
a sealing element attached to a first half-shell part facing the first housing part; and
a filter disposed within said first region, wherein one side of said filter seats against an inner region of a second half-shell part facing away from the first housing part and an other side of said filter seats against said sealing element, said filter thereby being loaded by an axial stress in an axial direction extending approximately perpendicularly to a dividing plane of said half-shell parts, wherein said axial stress is formed by joining of said half-shell parts and associated deformation of said sealing element by said filter.
